(19) United States
(12) Patent Application Publication
(10) Pub. No.: US 2016/0136060 A1
(43) Pub. Date: May 19, 2016

(54) WATER-BASED GEL COSMETIC COMPOSITIONS COMPRISING A POLYSACCHARIDE HYDROCOLLOID

(71) Applicant: L'OREAL, Paris (FR)

(72) Inventors: Christine Marie CRANE, Watchung, NJ (US); Angeles FONOLLA-MORENO, Scotch Plains, NJ (US)

(21) Appl. No.: 14/540,406

(22) Filed: Nov. 13, 2014

(51) Int. Cl. A61K 8/04; A61K 8/81; A61K 8/365; A61Q 1/10; A61K 8/89; A61K 8/891; A61K 8/73; A61K 8/19

(52) U.S. Cl. CPC A61K 8/042; A61K 8/737; A61K 8/8152; A61K 8/19; A61Q 1/10; A61K 8/89; A61K 8/891; A61K 8/73; A61K 8/365; A61K 2800/48; A61K 2800/872; A61K 2800/43; A61K 2800/594

(57) ABSTRACT

Disclosed is a water-based gel cosmetic composition comprising at least one polysaccharide hydrocolloid; at least one viscosity increasing agent different from the at least one polysaccharide hydrocolloid; at least one film forming polymer; at least one liquid fatty substance; optionally at least one nonionic surfactant having an HLB greater than or equal to about 15; water; at least one pH or cation adjuster; and optionally a pigment. Also disclosed are methods of making up the eyes with the composition of the invention as well as a method of making the composition.

### TECHNICAL FIELD

The present invention relates to water-based gel cosmetic compositions having a viscosity profile that mimics commercially available anhydrous gel eyeliners.

### BACKGROUND OF THE INVENTION

Cosmetic compositions in gel form are desirable as gels are easy to apply, afford a more consistent and precise coverage than liquids and are not as drying to the skin as powders. Water-based cosmetic compositions, in particular face and eyeliner compositions, are desirable as water based cosmetics can be easier to remove, are less likely to clog pores, may afford a less shiny and more natural look and feel, and are less expensive to process when compared to oil or solvent-based compositions. While aqueous cosmetic compositions are known, for example U.S. Pat. No. 6,641,823, these compositions are not gels. Most currently marketed gel eyeliners are anhydrous. They are not ideal as they contain hydrocarbon solvents (typically isododecane) and high volatile silicone fluids (such as cyclopentasiloxane). These anhydrous gels typically suffer from inconsistent wear, smudging and are difficult to remove. They typically are also less fresh and more uncomfortable to wear than water-based compositions. Additionally, water-based compositions are typically less costly to produce and offer a more sustainable environmental platform.

There remains need for a water-based gel cosmetic composition.

The current invention provides water-based gel cosmetic compositions that afford a comparable rheological profile to marketed anhydrous gel formulas. This affords the consumer the convenience of gels (e.g. easy control, precise product pick-up) having the desirable properties of both marketed anhydrous gel formulas (good glide, spreadability, coverage and intensity) and water-based cosmetics (freshness, ease of removal, good wear and comfort).

### BRIEF SUMMARY OF THE INVENTION

The present invention relates to a water-based gel cosmetic composition comprising (a)(i) at least one polysaccharide hydrocolloid; (a)(ii) at least one viscosity increasing agent different from the at least one polysaccharide hydrocolloid; (b) at least one film forming polymer; (c) at least one liquid fatty substance; optionally at least one nonionic surfactant having an HLB greater than or equal to about 15; water; at least one pH or cation adjuster; and optionally a pigment. In an embodiment the composition has a pH less than about 7.55.

The composition optionally may include other components appropriate for its intended use such as a non-gelling viscosity increasing agent, additional emollients, pigments, preservatives, neutralizers, vitamins, fillers, and the like. The compositions preferably do not include waxes.

Another embodiment of the invention relates to method of making up a keratinous substance, in particular the eyes, but applying to the eye lids the above-described composition.

Another embodiment of the invention relates to a method of improving at least one property selected from freshness, long wear, comfort, gentle application, color intensity, ease of removal, water and/or oil-resistance, adhesion, malleability, setting viscosity and transfer resistance of a gel eyeliner by incorporating in said eyeliner a synthetic non-associative thickening polymer, a synthetic associative thickening polymer, and a film forming polymer.

Another embodiment of the invention relates to a method of making the above-described cosmetic composition.

### DETAILED DESCRIPTION OF THE INVENTION

In the following description of the invention and the claims appended hereto, it is to be understood that the terms used have their ordinary and accustomed meanings in the art, unless otherwise specified. All concentrations are by weight percent on an active basis unless otherwise indicated.

“Aqueous phase” means the phase comprising water as well as such substances of a formulation which, due to their hydrophilic character, can be mixed in and/or dissolved in and/or dispersed in water. The aqueous phase of the composition according to the invention is advantageously a continuous aqueous phase. A “continuous aqueous phase” means that the composition has a conductivity, measured at 25° C., of greater than 23 microSiemens/cm, the conductivity being measured, for example, using an MPC227 conductimeter from Mettler Toledo and an Inlab 730 conductivity measuring cell.

“Easy removal” means the composition may be substantially removed with a non-harsh remover, such as water and/or with a water-based cleansing solution, and without excessive rubbing.

“Emulsifier or emulsifying surfactant” is a term of art that is well known to those skilled in the art. See, e.g. http://pharmlabs.unc.edu/labs/emulsions/agents.htm. It is a compound that has a hydrophilic part and a lipophilic part (“amphiphilic”) and facilitates the dispersion of two mutually insoluble phases, in this case the dispersion of a liquid fatty substance in water. A “surfactant” is a compound that lowers the surface tension (or interfacial tension) between two liquids or between a liquid and a solid. Surfactants may act as detergents, wetting agents, emulsifiers, foaming agents, and dispersants.

“High color intensity” means dramatic visual impact of the tint, in this instance, the blackness of the compositions and/or eye lids.

“HLB” as used herein means the hydrophilic-lipophilic balance of a molecule. It is the ratio between the hydrophilic part and lipophilic part of a molecule. This term is well known to those skilled in the art. See, e.g., “The HLB System: A Time-saving Guide to Emulsifier Selection” (Pub: ICI Americas Inc., 1984) and US2006/0217283 at [0053], both of which are herein incorporated by reference.

“Synthetic” means synthetically derived.

“Solids content” or “dry weight” as used herein refers to the weight of a polymer or active ingredient that has been dispersed in a liquid. It is the weight of the polymer (or active ingredient) either before it is dispersed in the liquid or after the liquid is evaporated/removed from the dispersion.

“Thickener” or “Viscosity increasing agent” is term that is well known to a skilled artisan. It means a compound or composition that increases the viscosity or resistance to flow of a composition to which it is added. See, Paint & Coating Testing Manual, 14^{th} Edition (J V Koleske Ed., 1995), pp. 268-288; and WO2011/076792. Typically, viscosity increasing agents increase the viscosity of an emulsion without any other significant change to the performance of the emulsion. Such compounds are described, for example, in U.S. Ser. Nos. 13/848,822 and 14/030,288, both of which are herein incorporated by reference. A synthetic viscosity increasing agent merely clarifies that the particular compound is synthetically derived as opposed to naturally occurring. Some thickeners are gelling agents (defined infra), others are mechanical thixotropic agents.

As used herein, all ranges provided are meant to include every specific range within, and combination of sub ranges between, the given ranges. Thus, a range from 1-5, includes specifically 1, 2, 3, 4 and 5, as well as sub ranges such as and 2-5, 3-5, 2-3, 2-4, 1-4, etc.

Unless otherwise specified herein, all percentages and ratios of components are by weight relative to the total weight of the final composition.
The following measurements referred to herein were made using the below-described procedures:

### Viscosity—Method A:

The viscosity of compositions was measured at 25° C. with a Rheomat 180 viscometer at 200 rpm (revolutions per minute) using a No. 3, 4 or 5 spindle. The viscosity was measured 10 minutes after switching on the rotation of the spindle and the values are reported in Pa·s (with the spindle denoted).

pH—Method B:

pH was determined using a Denver Scientific Ultrabasic pH Meter with a 3 mol/L KCl probe.

### Texture Analysis—Method C:

Texture properties of aged formulas were evaluated after sample preparation. Care was taken to ensure a flat surface. The analyzed samples for texture analysis were prepared by filling 50 gram FlackTek Inc., speed mixer containers to a total final weight of 54 grams (formula+container+lid) and speed mixed at 1000-2500 rpm for 1-4 minutes. Bottom of containers were then carefully tapped on a surface to ensure the formula surface was flat. Samples were allowed to rest at room temperature (20-25 degrees Celsius) for 72 hours. Penetration test was conducted on a TA.XT Plus Texture Analyzer with a cylindrical TA-56 steel probe (6 mm diameter). Settings: Test Mode: compression, Pre-test speed: 1 mm/sec, post-test speed: 2 mm/sec, test speed: 2 mm/sec, target mode: distance, distance: 8 mm, trigger force: auto, trigger force: 5 grams. After penetrating the sample the probe returned to its initial position. The curve generated is a plot of force (grams) as a function of time (seconds). When a 5 g surface trigger is attained the probe proceeds to penetrate to a depth of 8 mm. At this point (maximum +ve force), the probe returns to its original position at constant speed (e.g. 2.0 mm/s). The maximum +ve force (firmness, grams) and the +ve area under the curve (work of penetration, grams/sec) give an indication of the softness of the sample. The smaller the value, the softer is the sample. The negative region of the graph, produced on probe return, is an indication of the adhesive property of the product and/or as a result of a certain weight of sample that has adhered to the probe on return (Resistance to Probe, − grams/sec). (I. F. Alemeda, et. al., Intl. J. Pharma. 2006, 327, 73-77; H. Masmoudi, et al., Intl. J. Pharma. 2005, 289, 117-131). The results are reported in triplicate±standard deviation.

### Spreadability—Method D:

Spreadability was determined using a TTC Spreadability fixture, with a cone adapter and cup (TA-425) available from Texture Technologies on a TA.XT Plus Texture Analyzer. Settings: Compression mode with return to start option. Pre-test speed: n/a; test-speed: 3.0 mm/sec; post-test speed: 10 mm/s, distance 23 mm; trigger type: bottom, tare mode: auto; data acquisition: 200 pp. Female fixture was filled with approximately 5 grams of bulk prepared as described in the examples. Samples were tapped on solid surface to remove air pockets prior to run. Samples were kept at 25° C. The results are reported in triplicate±standard deviation.

### Centrifugal Stability Test—Method E:

Kimble/Chase 15 mL disposable glass centrifuge tubes were filled with 12 grams (8 cm high) of formula and they were placed in a Thermo Electron Corporation IEC CL30 Centrifuge at 2300 rpm (900 g) for 1 hour at 25° C. The samples tested by Method E were visually examined and physical changes in the appearance indicating demixing phenomena (creaming, foaming, or sedimentation) were measured by using a Fisher-scientific Caliper (06-664-16). If demixing was observed, it was recorded as “Yes,” meaning stable, or “no,” meaning not stable. “No” was assigned if less than or equal to 5% visible separation was observed (as determined by Equation 1 below) and/or a pellet was not observed at the base of the tube. If demixing was observed then it was noted as “Yes”. If a clear pellet formed, its height was determined by placing the test tube on its side, allowing a supernatant to reveal a lower meniscus level. The level was then marked and the height of pellet (hp), the height of supernatant (hs) or the height of other unidentified demixed phases (ho) was determined versus the total height of the formula in the tube (hT). The relative percent demixing was arbitrarily calculated using the following Equation 1:

% Demixing=(hs+ho)/ht*100%. Equation 1

In the gels of the invention, the combination of the polysaccharide hydrocolloid with a viscosity increasing agent different from the polysaccharide hydrocolloid, together with the film forming polymer, the liquid fatty substance, optionally the nonionic surfactant having and HLB greater than or equal to about 15, and a pH and/or a cation adjuster improves the firmness and “work of shear” of the compositions (as determined in a spreadability test using a Texture Analyzer equipped with a spreadability fixture using “Method D” supra) despite showing lower hardness as determined using method C. As used herein, “work of shear” means the total amount of force required to perform the shearing process.

In an embodiment, the invention relates to a water-based gel cosmetic composition comprising:
(a) a viscosity increasing system comprising (i) at least one polysaccharide hydrocolloid, and (ii) at least one viscosity increasing agent different from (a)(i);
(b) at least one film forming polymer;
(c) at least one liquid fatty substance;
(d) optionally at least one nonionic surfactant having an HLB greater than or equal to about 15;
(e) water;
(f) at least one pH adjuster or at least one cation adjuster, or a mixture of both; and
(g) optionally a pigment.

In an embodiment, the pH of the gels of the invention at 25° C. ranges from about 5 to less than about 7.55, typically from about 6 to about 7.15, most typically from about 6.5 to about 7.2, including all ranges and sub ranges therebetween.

In an embodiment the compositions of the invention comprise at least one nonionic surfactant.

In an embodiment the at least one viscosity increasing agent (a)(ii) is present in an amount from about 0.5% to about 3.5%, by weight, relative to the weight of the composition, and the ratio of the at least one polysaccharide hydrocolloid (a)(i) to the increasing agent (a)(ii) is about less than or equal to 4:1.

In an embodiment, the viscosity increasing agent (a)(ii) is selected from anionic polysaccharides, synthetic thickening agents, and mixtures thereof.
In another embodiment, the invention relates to a water-based gel cosmetic composition comprising:
   (a) a viscosity increasing system comprising (i) at least one polysaccharide hydrocolloid, (ii) at least one viscosity increasing agent selected from an anionic polysaccharide, (iii) optionally at least one synthetic viscosity increasing agent, and mixtures thereof;
   (b) at least one film forming polymer;
   (c) at least one liquid fatty substance;
   (d) at least one nonionic surfactant having an HLB greater than or equal to about 15;
   (e) water;
   (f) at least one pH adjuster, cation adjuster, or a mixture thereof; and
   (g) optionally a pigment;
   wherein ratio of the (a)(i) at least one polysaccharide hydrocolloid to the (a)(ii) viscosity increasing agent selected from an anionic polysaccharide [(a)(i)/(a)(ii)] is less than or equal to about 4:1; said composition having a viscosity greater than or equal to about 10 Pa·s (as determined by Method A), and a pH less than about 7.2.

In an embodiment, the polysaccharide hydrocolloid is selected from a gelling agent.

### Compositions Comprising a Neutral Polysaccharide

In an embodiment, the invention relates to a water-based gel cosmetic composition comprising:
(a) a viscosity increasing system comprising (i) at least one polysaccharide hydrocolloid selected from a galactomannan, and (ii) at least one viscosity increasing agent selected from an anionic polysaccharide;
(b) of at least one film forming polymer;
(c) at least one liquid fatty substance;
(d) at least one nonionic surfactant having an HLB greater than about 15;
(e) water;
(f) at least one pH modifier; and
(g) optionally a pigment;
said composition having a viscosity greater than or equal to about 10 Pa·s (as determined by Method A) and a pH less than about 7.1 (as determined by Method B).

In the foregoing embodiment, the at least one film forming polymer is selected from a styrene/acrylates copolymer, preferably together with a film forming silicone copolymer.

In another embodiment, the invention relates to a water-based gel cosmetic composition comprising:
(a) from about 1% to about 2% by weight of a viscosity increasing system comprising (i) from about 0.5% to about 1% by weight of at least one polysaccharide hydrocolloid selected from a galactomannan, and (ii) from about 0.5% to about 1% by weight at least one viscosity increasing agent selected from an anionic polysaccharide;
(b) from about 5% to about 20% by weight (as measured in amounts of polymer solids content) of at least one film forming polymer;
(c) from about 2% to about 20% by weight of at least one liquid fatty substance;
(d) from about 0.5% to about 3.5% by weight of at least one nonionic surfactant having and HLB greater than or equal to 15;
(e) from about 30% to about 60% by weight water;
(f) from about 0.1% to about 0.5% by weight at least one pH modifier; and
(g) optionally a pigment;
said composition having a viscosity greater than or equal to about 10 Pa·s (as determined by Method A) or B), and a pH less than about 7.1; the weight percent being relative to the total weight of the composition.

In an embodiment, the film former preferably comprises from about 5% to about 18% by weight (by solids content) of a styrene/acrylates copolymer and from about 1% to about 2% by weight (by solids content) of a film forming silicone copolymer, including all ranges and sub ranges therebetween.

In the foregoing embodiments, the galactomannan preferably is ceratonia siliqua gum (commercially available, for example, from Nissin Oillo).

In an embodiment, the compositions of the invention contain no wax, have a viscosity of from about 10 Pa·s to about 25 Pa·s as measured by Method A, and a firmness greater than about 1000 g as determined by Method D.

In a preferred embodiment the compositions of the invention comprise a pH adjuster in an amount from about 0.1% to about 0.5%, by weight.

### Compositions Comprising Homopolysaccharides

In another embodiment the composition the invention relates to a water-based gel cosmetic composition comprising:
(a) a viscosity increasing system comprising (i) at least one polysaccharide hydrocolloid selected from a homopolysaccharide, and (ii) at least one viscosity increasing agent selected from an anionic polysaccharide;
(b) of at least one film forming polymer;
(c) at least one liquid fatty substance;
(d) at least one nonionic surfactant having an HLB greater than or equal to about 15;
(e) water;
(f) at least one pH modifier, cation modifier, or a mixture thereof; and
(g) optionally a pigment;
said composition having a viscosity greater than or equal to about 10 Pa·s (as determined by Method A or B), and a pH less than about 7.

In an embodiment of the above composition, the at least one film forming polymer is selected from a styrene/acrylates copolymer, preferably together with a film forming silicone copolymer.

Also in the foregoing embodiment the composition preferably comprises a pH adjuster.

Also in the foregoing embodiment the composition preferably can be hot/warm poured into a mold.
In another embodiment the composition the invention relates to a water-based gel cosmetic composition comprising:
   (a) from about 0.5% to about 3.5% by weight of a viscosity increasing system comprising (i) from about 0.5% by weight to about 2.9% of at least one polysaccharide hydrocolloid selected from a homopolysaccharide, and either (ii) from about % to about 1.6% by weight of at least one viscosity increasing agent selected from an anionic polysaccharide, a synthetic thickener, or a mixture thereof;
   (b) from about 5% to about 20% by weight (as measured in amounts of polymer solids content) of at least one film forming polymer;
   (c) from about 5% to about 20% by weight of at least one liquid fatty substance;
   (d) from about 1.5% to about 3.5% by weight of at least one nonionic surfactant having an HLB greater than or equal to about 15;
   (e) from about 30% to about 60% by weight water;
   (f) from about 0.1% to about 0.5% by weight at least one pH modifier; and
   (g) optionally a pigment;
   (h) optionally at least one wax;
   said composition having a viscosity greater than or equal to about 10 Pa·s (as determined by Method A), and a pH less than about 7; the weight percent being relative to the total weight of the composition.

In the foregoing embodiment, the film former preferably comprises from about 5% to about 18% by weight of a styrene/acrylates copolymer and from about 1% to about 2% by weight of a film forming silicone copolymer, including all ranges and sub ranges therebetween.

Also in the foregoing embodiment, the composition comprises from about 0.1% to about 0.5% by weight of a pH adjuster, including all ranges and sub ranges therebetween, and has a pH less than about 7.0.

In the immediately foregoing embodiments the homopolysaccharide preferably is sclerotium gum (available for example from Alban Mueller) and contains no wax. Typically, composition has a viscosity form about 10 Pa·s to about 25 Pa·s as determined by Method A, and a firmness of greater than 1000 g as determined by spreadability using Method D.

### Compositions Comprising a Pseudoplastic Anionic Polysaccharide

In another embodiment the composition the invention relates to a water-based gel cosmetic composition comprising:
(a) a viscosity increasing system comprising (i) at least one polysaccharide hydrocolloid selected from a pseudoplastic anionic polysaccharide, (ii) at least one viscosity increasing agent selected from an anionic polysaccharide, and (iii) optionally at least one synthetic viscosity increasing agent different from (a)(ii);
(b) of at least one film forming polymer;
(c) at least one liquid fatty substance;
(d) optionally at least one nonionic surfactant having an HLB greater than or equal to about 15;
(e) water;
(f) at least one pH modifier, cation modifier, or a mixture thereof; and
(g) optionally a pigment;
said composition having a viscosity greater than about 25 Pa·s as measured by Method A, a pH less than about 7, said composition optionally further comprising and emulsifying agent.

In the foregoing embodiment, the at least one film forming polymer is preferably selected from a styrene/acrylates copolymer.

Also in the foregoing embodiment the composition comprises both a pH adjuster and a cation adjuster and has a pH less than about 7.0.

In another embodiment the composition the invention relates to a water-based gel cosmetic composition comprising:
(a) from about 0.7% to about 2% by weight of a viscosity increasing system comprising (i) from about 0.4% by weight to about 0.9% of at least one polysaccharide hydrocolloid selected from a pseudoplastic anionic polysaccharide, (ii) from about 0.2% to about 0.5% by weight of at least one viscosity increasing agent selected from a thickening anionic polysaccharide, and (iii) optionally from about 0.1% to about 0.6% by weight of at least one synthetic viscosity increasing agent different from (a)(ii);
(b) from about 5% to about 20% by weight (as measured by solid contents) of at least one film forming polymer;
(c) from about 5% to about 20% by weight at least one liquid fatty substance;
(d) from about 1.5% to about 4% of at least one nonionic surfactant having an HLB greater than or equal to 15;
(e) from about 30% to about 60% by weight water;
(f) from about 0.1% to about 0.5% by weight at least one pH modifier or at least one cation modifier, or a mixture of both; and
(g) optionally a pigment;
said composition having a viscosity greater than 25 Pa·s (as measured according to Method A), a pH less than about 7, and a firmness of greater than 1500 g (as measured according to Method D); wherein the weight percent is relative to the total weight of the composition.

In the foregoing embodiment, the film former preferably comprises from about 5% to about 18% by weight of a styrene/acrylates copolymer, including all ranges and sub ranges therebetween.

Also in the foregoing embodiment, the composition comprises from about 0.1% to about 0.25% by weight of a pH adjuster and from about 0.1% to about 0.25% by weight of a cation adjuster, including all ranges and sub ranges therebetween.

In the immediately foregoing embodiments, the thickening anionic polysaccharide preferably is xanthan gum (commercially available, for example, from Rhodia) or gellan gum (commercially available, for example, form FMC Corp.)

### Compositions Devoid of pH or Cation Modifiers

In another embodiment the composition the invention relates to a water-based gel cosmetic composition comprising:
(a) a viscosity increasing system comprising (i) at least one polysaccharide hydrocolloid selected from a neutral polysaccharide, (ii) at least one viscosity increasing agent selected from an anionic polysaccharide, and (iii) optionally at least one synthetic viscosity increasing agent different from (a)(ii);
(b) of at least one film forming polymer selected from a styrene/acrylates copolymer;
(c) at least one liquid fatty substance;
(d) at least one nonionic surfactant having an HLB greater than or equal to about 15;
(e) water;
(f) no pH adjuster or waxes; and
(g) optionally a pigment;
said composition have a viscosity less than about 5 Pa·s as determined by Method A using Spindle 3, and can be poured.

In the immediately preceding embodiment, the composition is fluid enough to be capable of being poured. In an embodiment, the composition is stable to sedimentation of pigments (that is, pigments do not separate or settle out) as measured by centrifugation using Method E.

In another embodiment the composition the invention relates to a water-based gel cosmetic composition comprising:
(a) a viscosity increasing system comprising (i) from about 0.2% to about 1.7% of at least one polysaccharide hydrocolloid selected from a neutral polysaccharide, (ii) from about 0.1% to about 0.2% of at least one viscosity increasing agent selected from an anionic polysaccharide, and (iii) optionally from about 0.1% to about 0.4% of at least one synthetic viscosity increasing agent different from (a)(ii);
(b) from about 5% to about 20% of at least one film forming polymer selected from a styrene/acrylates copolymer;
(c) from about 3% to about 20% at least one liquid fatty substance;
(d) from about 1.5% to about 4% of at least one nonionic surfactant having an HLB of greater than or equal to 15;
(e) water;
(f) no pH modifier or waxes; and
(g) optionally a pigment;
said composition have a viscosity less than about 5 Pa·s as determined by Method A using Spindle 3, and can be poured.

In the immediately preceding embodiment, the composition is fluid enough to be capable of being poured. In an embodiment, the composition is stable to sedimentation of pigments (that is, pigments do not separate or settle out) as measured by centrifugation using Method E.

In another embodiment the film-forming polymer is a silicone copolymer. In a particular embodiment the silicone copolymer is divinyldimethicone/dimethicone copolymer (and) C12-13 Pareth-23 (and) Pareth-3 (available for example from Dow Corning).

In another embodiment, the film forming polymer is selected from a combination of both a silicone copolymer and an acrylate copolymer. In a particular embodiment the silicone film-forming polymer is divinyldimethicone/dimethicone copolymer (and) C12-13 Pareth-23 (and) Pareth-3 (Dow Corning) and the acrylate copolymer is a styrene/acrylates copolymer, acrylates copolymer, styrene/acrylates/ammonium methacrylate copolymer or a mixture thereof.

In an embodiment wherein the polysaccharide hydrocolloid includes agarose, the composition typically has a pH lower than about 7.55.

In an embodiment wherein the polysaccharide hydrocolloid includes agarose, the film former preferably comprises both a styrene/acrylates/ammonium methacrylate copolymer, such as for example Syntran® 5760 from Interpolymer (INCI: styrene/acrylates/ammonium methacrylate copolymer (and) sodium laureth sulfate (and) caprylyl glycol, and styrene/acrylates copolymer, such as for example Joncryl 77 from BASF).

In another embodiment, the invention relates to a method of improving a property selected from freshness, long wear, comfort, gentle application, color intensity, ease of removal, water and/or oil-resistance, adhesion, malleability and transfer resistance of a gel eyeliner by incorporating in said eyeliner a at least one polysaccharide gelling agent, a film forming polymer, a pH and/or cation adjuster, and a liquid fatty substance.

In another embodiment, the invention relates to a method of making up a keratinous substance, in particular the eyes, with the above described cosmetic composition.

### Viscosity Increasing System (a)

The viscosity increasing system of the inventive compositions comprise at least one polysaccharide hydrocolloid (a)(i). The system optionally may include at least one viscosity increasing agent(a)(ii) that is different from (a)(i), and optionally also may include a second viscosity increasing agent (a)(iii) that is different for (a)(ii), or a mixture thereof.

The viscosity increasing agent (a)(ii) preferably is selected from an anionic polysaccharide.

The second viscosity increasing agent (a)(iii) preferably is selected from a synthetic viscosity increasing agent.

The viscosity increasing system (a) is present in the composition of the invention in an amount of from about 0.5% to about 3.5% by weight, typically from about 0.75% to about 3.45% by weight, more typically from about 0.8% to about 3.4% by weight.

The gel compositions of the invention may be in the form of an oil in water emulsion.

Polysaccharide Hydrocolloid (a)(i)

Some polysaccharide hydrocolloids can act as gellants (or gelling agents). Gelling agents are a subset of thickeners and are known to those skilled in the art. See, U.S. Pat. No. 8,562,961 (Bui et al., L'Oreal), U.S. Pat. No. 7,334,636 (Nguyen, Halliburton Energy Services, Inc.), U.S. Pat. No. 5,128,390 (Murphy et. al., Halliburton Company), all of which are herein incorporated by reference. Gellants are polymers that can form a gel on setting or upon acidification or treatment with a divalent or monovalent cation. A gel is solid, jelly like material that can have properties ranging from soft and week to hard and tough. Gels are substantially dilute and have no flow (meaning they are not pourable) in steady state. Gellants are known to provide structure to compositions through chain interactions or entanglements enabling the formation of gels, either alone or upon addition of a monovalent or divalent cation or by addition of a proton source. Y. Nitta et al., Gelation and gel properties of polysaccharides gellan gum and tamarind xyioglucan, J. Biol. Macromol. 5(3):47-52 (2005).

In the context of the current invention, the polysaccharide hydrocolloids are preferably gellants. Non-limiting examples of such gelling polysaccharide hydrocolloids are selected from polymers comprising polysaccharides, and derivatives thereof that contain one or more of the following monosaccharide units: galactose, mannose, glucoside, glucose, xylose, arabinose, fructose, glucuroic acid, or pyranosyl sulfate. These polysaccharides include, but are not limited to cellulose derivatives, homopolysaccharides, anionic polysaccharides and neutral polysaccharides.

Non-limiting examples of cellulose derivatives include hydroxyethylcellulose, carboxymethyl cellulose, carboxymethyl hydroxyethyl cellulose, and mixtures thereof. See, e.g. U.S. Pat. No. 4,579,670. In a particular embodiment the cellulose derivative is hydroxyethylcellulose.

In a preferred embodiment the polysaccharide hydrocolloid is selected from a homopolysaccharide, most preferably sclerotium gum.

In another preferred embodiment the polysaccharide hydrocolloid is selected from an anionic polysaccharide. Suitable anionic polysaccharides include carrageenan, gellan gum, and mixtures thereof. Additional examples are set forth in U.S. Pat. No. 6,197,319, which is herein incorporated by reference.

In another preferred embodiment the polysaccharide hydrocolloid is selected from a neutral polysaccharide selected from galactomannan gum, glucomannan gum, agarose, and mixtures thereof.

In an embodiment the polysaccharide hydrocolloid is a galactomannan gum, preferably ceratonia siliqua gum.

Other suitable polysaccharide hydrocolloids include guar gum, cationically substituted guar, hydroxypropyl guar, carboxymethyl hydroxypropyl guar and hydroxybutyl guar, sodium alginate, and mixtures thereof. See, e.g. U.S. Pat. No. 4,579,670.

In an embodiment, the at least one polysaccharide hydrocolloid (a)(i) is present in the composition of the invention in an amount of from about 0.5% to about 3.5%, typically from about 0.6% to about 3%, more typically from about 0.7% to about 2.5%, by weight, including all ranges and sub ranges therebetween, all weights being based on the total weight of the composition.

Viscosity Increasing Agents (a)(ii) and (a)(iii)

The viscosity increasing agent(s) (or thickeners) that may be useful in the practice of embodiments of the disclosure include those conventionally used in cosmetics such as polymers of natural origin and synthetic polymers. Viscosity increasing agents may be selected from, for example vegetable gums, liposoluble/lipodispersible polymers, salts, and mixtures thereof. Preferably the viscosity increasing agents are not waxes.

Representative viscosity increasing agents that may be used in the practice of embodiments according to the disclosure may be chosen from nonionic, anionic, cationic, and amphoteric polysaccharides and polymers (including acrylate-based polymers), polyamino compounds, amphiphilic polymers, and other viscosity modifiers such as cellulose-based thickeners (e.g., hydroxyethylcellulose, hydroxypropylcellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum), and gums derived from plant exudates (e.g., gum arabic, ghatti gum, karaya gum, gum tragacanth, iota-carrageenan gum, agar gum and carob gum),starches, cross-linked homopolymers of acrylic acid or of acrylamidopropane-sulfonic acid, associative polymers, non-associative thickening polymers, water-soluble thickening polymers, and mixtures of these.

(a)(ii) Viscosity Increasing Agent Different from the Polysaccharide

In a particular embodiment, the viscosity increasing agent (a)(ii) is selected from natural anionic thickening polysaccharides. Non-limiting examples of natural anionic thickening polysaccharides include xanthan gum, hyaluronic acid, gum arabic, gum karaya, gum tragacanthis, hyaluronan, chondroitin sulfate, and mixtures thereof.

Preferred natural anionic polysaccharide viscosity increasing agents are selected from xanthan gum (available from e.g. Rhodia, CP Kelco), hydroxyethylcellulose, and mixtures thereof.

Anionic thickening polysaccharides may be obtained from chemically modified neutral polysaccharides. Examples of anionic industrially derived polysaccharides include carboxymethylcellulose and carboxymethylstartch. Carboxymethylation (e.g. of pullulan or dextran) and sulfation (e.g. of pullulan or glucuronan) also yields other industrially derived anionic polysaccharides. See, Z. Souguir et al., “Anionic Polysaccharide Hydrogels with Charges Provided by the Polysaccharide or the Crosslinking Agent,” Drug Delivery Letters, 2012, 2:4 pp 1-11; and “Polysaccharides (Sugras, Gums) Used in Cosmetics, www.makingcosmetics.com.

The at least one viscosity increasing agent (a)(ii) that is different from the polysaccharide hydrocolloid (a)(i) is present in the composition of the invention in an amount of from about 0.1% to about 3%, typically from about 0.2% to about 2.5%, more typically from about 0.3% to about 1.5%, including all ranges and sub ranges therebetween, all weights being based on the total weight of the composition.

Synthetic Viscosity Increasing Agent (a)(iii)

In another embodiment, at least an additional viscosity increasing agent (a)(iii) is also used. Preferably, this additional viscosity increasing agent is selected from a synthetic viscosity increasing agent. Such agents are known in the art of cosmetics formulations. See, e.g. US 2013/0243715; US 2004/0028637, both of which are herein incorporated by reference. Non-limiting examples of useful synthetic viscosity increasing agents include synthetic polymers such as polyacrylic acid (available commercially as Carbomers) and acrylates copolymers such as sodium polyacrylate and polyacryloyldimehtyl taurate, and mixtures of these.

Examples of particular synthetic viscosity increasing agents include ammonium polyacryloyldimethyl taurate, ammonium acryloydimethyltaurate/steraeth-25 methacrylate crosspolymer, ammonium acryloydimethyltaurate/steareth-8 methacrylate copolymer, sorbitol/sebacic acid copolymer behenate, ethylenediamines/stearyl dimer dilinoleate copolymer, acrylamide/sodium acryloyldimethyltaurate copolymer, and mixtures thereof.

In a particular embodiment the synthetic thickening agent is selected from ammonium polyacryloyldimethyl taurate (available from e.g. Clariant), ammonium acryloydimethyltaurate/steraeth-8 methacrylate copolymer (available from e.g. Clariant), acrylamide/sodium acryloyldimethyltaurate copolymer (such as acrylamide/sodium acryoyldimethyltaurate copolymer (and) isohexadecane (and) polysorbate 80 (available from Seppic), and mixtures thereof.

In embodiments wherein a synthetic viscosity increasing agent is present in the compositions of the invention, it is typically present in an amount of from about 0.1% to about 1%, preferably from about 0.15% to about 0.6%, more particularly from about 0.2% to about 0.5%, by weight, including all ranges and sub ranges therebetween, all weights being based on the total weight of the composition.

### Film-Forming Polymer (b)

As used herein, the terms “film-forming polymer,” “film former,” “film-forming agent” and variations thereof mean a polymer capable of, by itself or in the presence of an auxiliary film-forming polymer, forming a continuous film that adheres to a support and especially to keratin materials, for instance the eyes.

The film-forming polymers that may be used in the cosmetic compositions disclosed herein include, for example, acrylate copolymers, styrene/acrylate copolymers, acrylamide/acrylate copolymers, polyurethanes, silicone resins and block silicone copolymer particles, and other resins, and derivatives thereof and mixtures thereof.

In accordance with various exemplary embodiments, acrylate copolymers may be chosen from copolymers comprising two or more monomers chosen from acrylic acid, methacrylic acid, and their simple esters, for example, lower alkyl esters such as methyl, ethyl, and ethylhexyl esters. Such copolymers are discussed, for example in US 2013/0039874, which is herein incorporated by reference.

By way of non-limiting example only, acrylate copolymers may be chosen from styrene acrylates/copolymers, ammonium acrylates copolymers, ethyl acrylates copolymers, acrylates/ethylhexylacrylate copolymers, acrylates/octylacrylates copolymers, alkyl (meth)acrylates copolymers, acrylates/C₁₂-C₂₂ alkylmethacrylate copolymers, ethylacrylate/methacrylic acid copolymer, and t-butyl acrylate/ethyl acrylate/methacrylic acid copolymer. Exemplary commercial acrylate copolymers include, but are not limited to, ALLIANZ™ OPT sold by Ashland Specialty Ingredients; COVACRYL A15 and COVACRYL E14 sold by Sensient Cosmetic Technologies LCW; DAITOSOL 4000 SJT, DAITOSOL 5000 AD, DAITOSOL 5000 SJ, KOBOGUARD® 50A, and KOBOGUARD® 50N sold by Kobo Products, Inc.; DERMACRYL® AQF, YODOSOL 32A707, YODOSOL GH15, YODOSOL GH32, YODOSOL GH33, YODOSOL GH34, YODOSOL GH35, YODOSOL GH800, and YODOSOL GH810 sold by AkzoNobel; LUVIFLEX® SOFT, LUVIMER® 36D, and LUVIMER® 100P sold by BASF; and NEOCRYL XK-90 sold by Neoresins, Inc.

The film forming polymer may also be chosen from polyacrylates such as polyacrylate-21, and polyacrylate-15, and acrylates copolymer.

In another embodiment the at least one film forming polymer can be selected from a lipophilic polymer, such as for example, poly C₁₀₋₃₀ alkylacrylates (available as Intelimer® IPA 13-1 from Air Products).

### Latex Film Formers

The film-forming polymer may also be chosen from latex film forming polymers such as polyacrylate latex, polyurethrane latex, and their copolymers. Such polymers are discussed, for example, in e.g. US 2013/0084255 and US 2013/0084256, both of which are herein incorporated by reference.

Suitable examples of latex polymers for use in the present invention are ethylhexyl acrylate/hema copolymer (and) acrylates/diethylaminoethyl methacrylate/ethylhexyl acrylate copolymer (Syntran® PC 5775), styrene/acrylates/ammonium methacrylate copolymer (Syntran® 5760, Syntran® 5009, Syntran® PC5620), polyacrylate-21 (and) acrylates/dimethylaminoethyl methacrylate copolymer (Syntran® PC5100, Syntran® PC5776, Eudragit E 100, Jurymer ET-410C), styrene/acrylates/ammonium methacrylate copolymer (Syntran® 5009 CG), olefin/acrylate grafted polymer (and) sodium laureth sulfate (and C12-15 SEC-pareth 15 (Syntran® EX108), acrylates copolymer (Aculyn® 33A Polymer, Avalure® Ace 210/120/315 Acrylic Copolymer, Carbopol Aqua SF-1 ®Polymer, Daitosol® 500 AD, Coatex® Co 633, Eliclear® 380/700/4U, Eudragit® L 100, Joncryl® 85, Luviflex® Soft), acrylates/ethylhexyl acrylate copolymer (Daitosol® 5000SJ, Daitosol® 4000SJT, MJA PS34-21, SDP-001). The Syntran® polymers are commercially available from the supplier Interpolymer Corp.

Suitable examples of latex polymers are polyurethane-35, polyurethane-35,and polyurethane-35.

In an embodiment, the latex polymer is an acrylate latex polymer, in particular styrene/acrylate copolymers. Non-limiting examples of commercially available styrene/acrylate copolymers include, but are not limited to, DAITOSOL 5000 STY sold by Kobo Products, Inc.; JONCRYL® 77 (styrene/acrylates copolymer, sold by BASF); NEOCRYL BT-62 sold by Neoresins, Inc.; RHOPLEX™ P-376 and UCAR™ DL 432S sold by Dow Chemical Company; and YODOSOL GH41 and YODOSOL GH840 sold by AkzoNobel.

In further exemplary embodiments, acrylamide/acrylate copolymers may be chosen from acrylic acid/ethyl acrylate/t-butyl acrylamide copolymer, acrylates/octylacrylamide copolymer, and octylacrylamide/acrylates/methacrylates copolymer. Exemplary commercial acrylamide/acrylate copolymers include, but are not limited to AMPHOMER® LV-71 and DERMACRYL® 79 sold by AkzoNobel and ULTRAHOLD® STRONG sold by BASF.

According to additional exemplary embodiments, polyurethanes may be chosen from polyurethane-32, polyurethane-34, and polyurethane-35. Exemplary commercial polyurethanes include, but are not limited to, products sold by Bayer under the trade name BAYCUSAN®, such as BAYCUSAN® C1000, BAYCUSAN® C1001, BAYCUSAN® C1003, and BAYCUSAN® C1004.

In at least one exemplary embodiment, the latex film former may be chosen from blends comprising a combination of latex film formers, including, for example, a blend of any of the above-mentioned film formers. By way of non-limiting example, a latex film forming blend useful according to the disclosure may comprise (1) at least one random styrene acrylate copolymer or derivatives thereof, and at least one acrylate copolymer or derivatives thereof, or (2) at least two random styrene acrylate copolymers or derivatives thereof.

In an embodiment the film former is selected from styrene/acrylates/ammonium methacrylate copolymers sold by Interpolymer Corporation, in particular SYNTRAN® 5760 (styrene/acrylates/ammonium methacrylate copolymer (and) sodium laureth sulfate (and) caprylyl glycol); SYNTRAN® 5775 (acrylates/ethylhexyl acrylate/hema copolymer (and) acrylates/diethylaminoethylmethacrylate/ethylhexyl acrylate copolymer (and) isodeceth-6 (and) caprylyl glycol (and) sodium laureth sulfate); SYNTRAN® Ex 108 (olefin/acrylate grafted polymer (and) sodium laureth sulfate (and) C12-15 SEC-pareth 15); and SYNTRAN® 108 GC (olefin/acrylic grafted emulsion).

According to at least certain exemplary embodiments of the disclosure, the at least one latex film former may be chosen from those having a glass transition temperature (Tg) ranging from about −15° C. to about 90° C., such as from about 0° C. to about 50° C.

In a particular embodiment the styrene acrylates film former is selected from styrene/acrylates/ammonium methacrylate copolymer (such as (styrene/acrylates/ammonium methacrylate copolymer (and) sodium laureth sulfate (and) caprylyl glycol), SYNTRAN® 5760 from Interpolymer), and styrene/acrylates copolymer (such as JONCRYL 77 from BASF), and mixtures thereof.

### Silicone Resins

In accordance with other exemplary embodiments, silicone resins and copolymers may also be used as a film forming agent. In a particular embodiment, the at least one firm forming polymer is a silicone copolymer.

Film-forming silicone resins and copolymers are described, for example, in U.S. Pat. No. 7,790,148 (L'Oreal), U.S. Pat. No. 7,094,842 (L'Oreal), US2011/0189,117 (L'Oreal), US2010/0297050 (L'Oreal), US 2007/0093619 and 2006/0013791, 2005/0201961, all of which are herein incorporated by reference.

Exemplary film forming silicone resins are cross-linked polyorganosiloxane polymers. The nomenclature of silicone resins is known under the name “MDTQ”, the resin being described as a function of the various monomeric siloxane units it includes, each of the letters “MDTQ” characterizing one type of unit. Examples of commercially available polymethylsilsesquioxane resins that may be mentioned are those that are sold by the supplier Wacker under the reference Resin MK such as Belsil® PMS MK, by the supplier SHIN-ETSU under the references KR-220L, and Silform® Flexible Resin from the supplier Momentive.

Examples of commercially available polypropylsilsesquioxane resins useful in the invention include those sold under the reference DC®670 by the supplier Dow Corning, and Silform® Flexible Resin from the supplier Momentive.

Examples of a polyphenylsilsesquioxane resins useful in the invention include those available from Wacher.

Siloxysilicate resins that may be used include trimethylsiloxysilicate resins (TMS®) such as those sold under the reference SR1000 by the supplier Momentive Performance Materials or under the reference TMS® 803 by the supplier Wacker. Trimethylsiloxysilicate resins are also available in a solvent such as cyclomethicone, sold under the name “KF-7312J” by the supplier Shin-Etsu, or “DC® 749”, “DC® 593” by the supplier Dow Corning.

Pressure-sensitive adhesive silicone copolymers are also herein contemplated. Such copolymers are available, for example from Dow Corning under the reference BIO-PSA and described in U.S. Pat. No. 5,162,410, which is herein incorporated by reference.

Silicone copolymers derived from the reaction of a silicone resin such as those described above and of a diorganosiloxane such as that described in the document WO 2004/073626 are also contemplated.

### Block Silicone Copolymer Particles

Block silicone copolymer particles are also useful in preparing films according to the invention. These silicone compounds are described in U.S. Pat. No. 7,094,842, which is herein incorporated by reference. The silicone copolymer constituting the globules or particles in dispersion in the aqueous phase is a substantially linear block copolymer, that is to say a non-cross-linked copolymer, obtained by chain extension and not by crosslinking.

The aqueous dispersion of particles of block copolymer is a silicone-in-water emulsion (Sil/W) wherein the oily globules are constituted from a silicone of high viscosity, so that these globules seem to form as “soft particles”.

The composition may comprise dispersions of one or more types of substantially linear block silicone copolymer. These block silicone copolymers are present in the composition of the invention in concentrations, as active material, which may vary widely depending on the other ingredients of the composition and the desired aim. The concentration, as active material (A.M.), of block silicone copolymer preferably ranges from 0.01 to 15% by weight, even better from 0.1 to 10% and better still from 0.5 to 5% by weight relative to the total weight of the composition.

The size of the block silicone copolymer particles vary. Preferably, in the present invention, the silicone copolymer particles generally have a number-average size of less than or equal to 2 microns, and preferably of less than or equal to 1 micron.

The aqueous dispersions of substantially linear block silicone copolymer particles used according to the invention may be chosen in particular from those described in the document EP-A-874017. According to this document, it is possible in particular to obtain the silicone copolymers constituting these particles by chain extension reaction, in the presence of a catalyst, from at least:
(a) one polysiloxane (i) having at least one reactive group and preferably one or two reactive groups per molecule; and
(b) one organosilicone compound (ii) which reacts with the polysiloxane (i) by chain extension reaction.

In a particular embodiment, the polysiloxane (i) is chosen from a compound of formula (V):

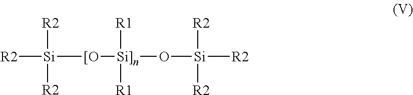

wherein R₁ and R₂, independently of each other, represent a hydrocarbon group having from 1 to 20 carbon atoms and preferably from 1 to 10 carbon atoms, such as methyl, ethyl, propyl or butyl, or an aryl group such as phenyl, or a reactive group, n is an integer greater than 1, provided that there is on average between one and two reactive groups per polymer.

As used above in defining formula (V), the expression “reactive group” is understood to mean any group capable of reacting with the organosilicone compound (ii) to form a block copolymer. As reactive groups, there may be mentioned hydrogen; aliphatically unsaturated groups and in particular vinyl, allyl or hexanyl groups; the hydroxyl group; alkoxy groups such as methoxy, ethoxy or propoxy; alkoxyalkoxy groups; the acetoxy group; amino groups, and mixtures thereof. Preferably, more than 90%, most preferably more than 98% of reactive groups are at the chain end, that is to say that the radicals R₂ generally constitute more than 90% and even 98% of the reactive groups.

Preferably, n is such that the polysiloxanes have a viscosity ranging from about 1 to 1×10⁶ mm²/sec at 25° C. n may be for example an integer ranging from about 5 to 30, preferably from 10 to 30 and better from 15 to 25.

The polysiloxanes of formula (V) are substantially linear polymers, that is to say containing few branches, and generally less than 2 mol % of the siloxane units. Moreover, the groups R₁ and R₂ may be optionally substituted with amino groups, epoxy groups, groups containing sulfur, silicon or oxygen.

Preferably, at least 80% of the groups R₁ are alkyl groups and even better methyl groups.

Preferably, the reactive group R₂ at the chain end is an aliphatically unsaturated group and in particular a vinyl group.

As polysiloxanes (i), there may be mentioned in particular dimethylvinylsiloxypolydimethylsiloxane, a compound of formula (V) in which the radicals R₁ are methyl radicals, and, at the chain end, the radical R₂ is a vinyl radical while the other two radicals R₂ are methyl radicals.

The organosilicone compound (ii) may be chosen from the polysiloxanes of formula (V) or compounds acting as chain extension agent. If it is a compound of formula (V), the polysiloxane (i) will contain a first reactive group and the organosilicone compound (ii) will contain a second reactive group which will react with the first. If it is a chain extension agent, it may be a silane, a siloxane (disiloxane or trisiloxane) or a silazane. Preferably, the organosilicone compound (ii) is a liquid organohydrogenpolysiloxane of formula (VI):

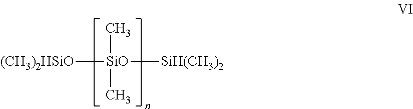

wherein “n” is an integer greater than 1 and preferably greater than 10, and for example ranging from 5 to 30, preferably from 10 to 30, and better from 15 to 25. According to a particular embodiment of the invention, “n” is equal to 20.

The block silicone copolymers used according to the invention are advantageously free from oxyalkylenated groups, especially free from oxyethylenated and/or oxypropylenated groups.

The catalyst of the reaction between the polysiloxane and the organosilicone compound may be chosen from metals and in particular from platinum, rhodium, tin, titanium, copper and lead. It is preferably platinum or rhodium.

The dispersion of silicone copolymer particles used according to the invention may in particular be obtained, for example, by mixing (a) water, (b) at least one emulsifier, (c) the polysiloxane (i), (d) the organosilicone compound (ii) and (e) a catalyst. Preferably, one of the constituents (c), (d) or (e) is added last to the mixture so that the chain extension reaction only starts in the dispersion.

As emulsifiers which may be used in the method of preparation described above for obtaining the aqueous dispersion of particles, there may be mentioned non-ionic or ionic (anionic, cationic or amphoteric) emulsifiers. They are preferably non-ionic emulsifiers which may be chosen from polyalkylene glycol ethers of a fatty alcohol, containing from 8 to 30 carbon atoms and preferably from 10 to 22 carbon atoms; polyoxyalkylenated and in particular polyoxyethylenated alkyl esters of sorbitan, where the alkyl radical contains from 8 to 30 carbon atoms and preferably from 10 to 22 carbon atoms; polyoxyalkylenated and in particular polyoxyethylenated alkyl esters, where the alkyl radical contains from 8 to 30 carbon atoms and preferably from 10 to 22 carbon atoms; polyethylene glycols; polypropylene glycols; diethylene glycols; and mixtures thereof. The quantity of emulsifier(s) is generally from 1 to 30% by weight relative to the total weight of the reaction mixture.

The emulsifier used to obtain the aqueous dispersion of particles is preferably chosen from polyethylene glycol ethers of fatty alcohols and mixtures thereof, in particular polyethylene glycol ethers of alcohols containing 12 or 13 carbon atoms or from 2 to 100 oxyethylenated units and preferably from 3 to 50 oxyethylenated units, and mixtures thereof. There may be mentioned, for example, C₁₂-C₁₃ Pareth-3, C₁₂-C₁₃ Pareth-23 and mixtures thereof.

According to a particular embodiment of the invention, the dispersion of particles of silicone copolymer is obtained from dimethylvinylsiloxypolydi-methylsiloxane (or divinyldimethicone) as compound (i), and from the compound of formula (II) with preferably n=20, as compound (ii), preferably in the presence of a platinum-type catalyst, and the dispersion of particles is preferably obtained in the presence of C₁₂-C₁₃ Pareth-3 and C₁₂-C₁₃ Pareth-23 as emulsifiers.

A non-limiting example of dispersion of particles of silicone copolymer is divinyldimethicone/dimethicone copolymer (and) C12-13 pareth-23 (and) C12-13 pareth-3 (INCI name, commercially available as HMW 2220 from Dow Corning; containing about 60-% by weight of divinyldimethicone/dimethicone copolymer).

### Particular Embodiments of Film-Forming Polymers

In particular exemplary embodiments, the film forming-polymer is a silicone film forming polymer selected from siloxysilicate resins, in particular trimethylsiloxysilicate resin.

In another particular embodiment, the at least one silicone film forming polymer is selected from polypropylsilsesquioxane resin, acrylates/dimethicone copolymers, cyclohexasiloxane, cyclopentasiloxane, cetyl PEG/PPG-10/1 dimethicone, Nylon-611/Dimethicone copolymer, polyglycerin modified branched silicone crosspolymers, divinyldimethicone/dimethicone copolymers, and mixtures thereof.

In another embodiment the film former is selected from an acrylates copolymer, such as acrylates/ethylhexyl acrylate copolymer, commercially available from KOBO under the name DIATOSOL®.

In another particular embodiment the film former is selected from a styrene/acrylates copolymer.

In another embodiment the film former is selected from both an acrylate copolymer, such as styrene/acrylates copolymer, and a silicone copolymer, such as divinyldimethicone/dimethicone copolymer.

In another embodiment the film forming polymer may also be selected from a natural resin/polymer, such as shellac and its derivatives.

The active polymer content of the at least one film-former (c) is present in the composition of the invention in an amount of from about 0.5% to about 20%, preferably from about 1% to about 18%, more preferably from about 2% to about 15%, including all ranges and sub ranges therebetween, by weight, all weights being based on the total weight of the composition.

In an embodiment wherein the film former is acrylate-based, the amount of active polymer film former in the composition is from about 1% to about 20%, typically from about 4% to about 18%, more typically from about 5% to about 15%, including all ranges and sub ranges therebetween, by weight, all weights being based on the total weight of the composition.

In another embodiment wherein the film former is silicone-based, the amount of active film former in the composition is from about 0.1% to about 3%, typically from about 0.5% to about 2.5%, including all ranges and sub ranges therebetween, by weight, all weights being based on the total weight of the composition. When the film former is a silicone resin, the amount of the silicon resin in the composition is typically from about 2% to about 15%, most typically from about 5% to about 10%, including all ranges and sub ranges therebetween, by weight, all weights being based on the total weight of the composition. When the film former is a silicone block copolymer (for example divinyl/dimethicone cross polymer), the active component is present in the composition in an amount from about 0.1% to about 5%, most typically about from about 1% to about 2.5%, including all ranges and sub ranges therebetween, by weight, all weights being based on the total weight of the composition.

In another embodiment wherein the film former includes both an acrylate-based polymer and a silicone-based block polymer, silicone-based block copolymer (by active solid polymer) is present in an amount of from about 0.1% to about 3%, typically about 1.5%, and the acrylate-based polymer is present in an amount of from about 0.1% to about 20%, more typically about 5% to about 15%, including all ranges and sub ranges therebetween, by weight, all weights being based on the total weight of the composition.

In another embodiment the film former includes a silicone resin copolymer. In this embodiment, the silicone resin copolymer is present in an amount of from 2% to about 15%, more typically from about 5% to about 10%, all weights being based on the total weight of the composition.

### Liquid Fatty Substance (c)

The compositions according to the invention contain a liquid fatty substance.

The liquid fatty substance may be selected, for example, from organic solvents and volatile and/or non-volatile oils, and mixtures thereof. These oils include low viscosity oils (having a viscosity from about 5 to about 10 centipoise) and high viscosity oils (having a viscosity of from about 100 to about 10,000 centipoise), and mixtures thereof. These fatty substances are liquid at room temperature. Such liquid fatty substances include silicones, alkoxylated silicones, oils, ethoxylated oils, fats, esters, transesters, hydrocarbons, quats and mixtures thereof.

Representative suitable solvents include non-polar volatile hydrocarbon-based oils include isodecane and isododecane, and for example, the oils sold under the trade names Isopar™ or Permythyl®. Preferably, the volatile oils have a flash point of at least 40° C.

Other exemplary organic solvents are non-volatile solvents which include polyalphaolefins such as hydrogenated polydecene, hydrogenated C6-14 olefin polymers and polydecene.

Natural oils may also be used so long as they are physiologically acceptable. Such oils include hydrocarbon-based plant oils with high triglyceride content such as sweet almond oil, avocado oil, olive oil, candlenut oil, vitamin E oil, jojoba oil, apricot oil, castor oil, lanolin and the like.

Volatile and non-volatile silicone oils, may also be used. Such oils are described, for example in US 2011/0293550, which to the extent required, is herein incorporated by reference. Suitable silicone oils include, for instance, volatile or non-volatile polymethylsiloxanes (PDMS) with a linear or cyclic silicone chain, which are liquid or pasty at room temperature, including cyclopolydimethylsiloxanes (cyclomethicones) such as cyclohexasiloxane; polydimethyl-siloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenyl-siloxanes, diphenyl dimethicones, diphenylmethyl-diphenyltrisiloxanes or 2-phenylethyl trimethylsiloxy silicates, and polymethylphenylsiloxanes (trimethyl pentaphenyl trisiloxane); and dimethicone fluids having viscosity values of equal to or greater than 300 cPs; amodimethicone, dimethicone, dimethiconol, cyclemethicone, phenyltrimethicone, aminopropyl phenyltrimethicone, cetyl dimethicone, alkyl dimethicone, potassium dimethicone PEG-7 pantheyl phosphate; and mixtures thereof.

The fatty substance may also be selected from non-silicone oils such as hydrogenated polyisobutene; and fatty esters such as isostearyl hydroxy stearate, glyceryl ethylhexanoate/stearate/adipate, pentaerythrityl tetraethylhexanoate, isononyl isononanoate, and isopropyl lauroyl sarcosinate; and mixtures thereof.

The liquid fatty substance may also be selected from capric/caprylic triglyceride, octyl Palmitate, Isopropyl palmitate, isopropyl myristate, mineral oil, petrolatum, and mixtures thereof.

In a particular embodiment the liquid fatty substance is selected from dimethicone (such as dimethicone (and) dimethicone/PEG-10/15 crosspolymer commercially available from Shin Etzu; dimethicone (and) dimethiconol commercially available from Bluestar), phenyl trimethicone, PEG-10 dimethicone, dimethiconol, caprylyl methicone, bis-diglyceryl polacyladipate-2, and mixtures thereof.

The liquid fatty substance (d) is present in the composition of the invention in an amount of from about 1% to about 20%, preferably from about 2% to about 18%, more particularly from about 3% to about 15%, by weight, including all ranges and sub ranges therebetween, all weights being based on the total weight of the composition.

### Nonionic Surfactant (d)

The cosmetic compositions of the invention comprise at least one nonionic surfactant.

In a particular embodiment, the nonionic surfactant has an HLB equal to or greater than 15.

Nonionic surfactants useful in the cleansing compositions disclosed herein can be selected from; alkyl polyglucosides; ethylene glycol, propylene glycol, glycerol, polyglyceryl esters and their ethoxylated derivatives (herein jointly referred to as “glycol esters”); and mixtures of the foregoing.

Alkyl polyglucosides useful in the compositions of the invention include those having the following formula:

R¹—O—(R²O)n-Z(x) (XXX)

wherein

R¹ is an alkyl group having 8-36 carbon atoms;

R² is an ethylene or propylene group;

Z is a saccharide group with 5 to 6 carbon atoms;

n is an integer from 0 to10; and

x is an integer from 1 to 5.

Non-limiting examples of nonionic alkyl poly glucosides useful in the compositions of the invention include lauryl glucoside, octyl glucoside, decyl glucoside, coco glucoside, sucrose laurate, caprylyl/capryl glucoside, and mixtures thereof. Typically, the at least one alkyl polyglucoside compound is selected from the group consisting of lauryl glucoside, decyl glucoside, coco glucoside, and mixtures thereof.

In a particular embodiment the nonionic surfactant is an alkyl poly glucoside selected from decyl glucoside and coco glucoside, and mixtures thereof.

Non-limiting examples of glycol esters useful in the compositions of the invention include those described in M. R. Porter et al., Handbook of Surfactants, Ch. 7, §7.12, pp. 231-235 (2^{nd} Ed. 1994), which is herein incorporated by reference. Preferred glycol esters have HLB values greater than or equal to 15, such as between about 15 and about 18. Particular glycol esters useful in the compositions of the invention include polysorbate-40.

Further non-limiting examples of nonionic surfactants having an HLB greater than or equal to 15 that may be used in the compositions of the invention include polyoxyethylene (100) stearyl ether (such as BRIJ® 100 from Aldrich), PEG-40 hydrogenated castor oil, PEG-60 hydrogenated castor oil, PEG-25 hydrogenated castor oil, polyoxyethylene (20) oleyl ether (such as BRIJ® O20 from Sigma), polyethylene glycol hexadecyl ether polyoxyethylene (20) cetyl ether (such as BRIJ® 58 from Sigma), polyethylene (100) stearyl ether (such as BRIJ® 100 from Sigma), polyethylene glycol octadecyl ether polyoxyethylene (20) stearyl ether (such as BRIJ® S20 from Sigma), polyoxyethylene (40) nonylphenyl ether, branched (such as IGEPAL® CO-890 from Sigma), polyoxyethylene (150) dinonylphenyl ether polyoxyethylene, dinonylphenyl and nonylphenyl ethers, branched (such as IGEPAL® DM-970 from Sigma), polyethylene glycol sorbitan monolaurate polyoxyethylenesorbitan monolaurate (such as TWEEN® 20 from Aldrich), polyoxyethylenesorbitan monopalmitate (such as TWEEN® 40 from Aldrich), polyethylene glycol sorbitan monostearate polyoxyethylene sorbitan monostearate (such as TWEEN® 60 from Aldrich), polysorbate 80 (such as TWEEN® 80-LC-CQ from Croda), and mixtures thereof.

In a particular embodiment, the non-ionic surfactant having an HLB greater than or equal to 15 is polysorbate 80.

Some of the foregoing surfactants may also be referred to as “emulsifying” surfactants. Emulsifying surfactants are known to those skilled in the art.

In a particular embodiment, the compositions of the invention may also include additional emulsifying surfactants/dispersants. Such additional emulsifying surfactants may be selected from, for example, sodium laureth sulfate, PPG-7, sorbitan oleate, C12-13 Pareth 3, C12-13 parareth-23, and mixtures thereof.

The at least one nonionic surfactant (d) may be used in an amount of from about 0.1% to about 5%, such as from 0.5% to about 4%, more particularly from about 0.6% to about 3.5%, including all ranges and sub ranges therebetween, by weight, relative to the weight of the final composition.

### Water (e)

The compositions of the invention also comprise water in an amount ranging from about 25% to about 60%, preferably from about 28% to about 57%, most typically from about 30% about 55%, including all ranges and sub ranges therebetween, by weight, relative to the total weight of the compositions.

### Other Solvents (Optional

The compositions of the invention may include additional solvents. In particular, the aqueous phase may include at least one organic solvent that is water-miscible. Non-limiting examples of suitable organic solvents include C₁₋₄ alkanols, such as ethanol, isopropanol, t-butyl alcohol; glycerol; glycols and glycol ethers such as 2-butoxyethanol, propylene glycol, butylene glycol, monomethyl ether of propylene glycol, monethyl ether and monomethyl ether of diethylene glycol, aromatic alcohols such as benzyl alcohol and phenoxyethanol; analogous products and mixtures of the foregoing products.

Other solvents include caprylic/capric acid triglycerides (such as those sold under the trade name Miglyol®, isohexadecane, and mixtures thereof.

In addition to water, the compositions of the invention may comprise a solvent in an amount ranging from about 0.1% to about 10%, preferably from about 3% to about 6%, including all ranges and sub ranges therebetween, by weight, relative to the total weight of the compositions.

pH Modifier and/or Cation Modifier (f) (Optional)

The compositions of the invention optionally may comprise a pH modifier (also referred to as pH adjusters), or a cation modifier (or adjuster), or a combination of both.

pH modifiers/neutralizing agents and cation modifiers useful in cosmetic formulations are known. See, e.g. International Cosmetic Ingredient Dictionary and Handbook (9^{th} ed. 2002, and subsequent editions). Non-limiting examples of pH modifiers useful in the invention include acetic acid, ascorbic acid, citric acid, sodium citrate, tartaric acid, ammonium hydroxide, and mixtures thereof. In a preferred embodiment, the pH adjuster is selected from citric acid, ascorbic acid, sodium citrate, ammonium hydroxide.

When present in the compositions of the invention, the pH modifier is present in an amount such as to result in a final pH of the composition of from about 5 to less than about 7.55, typically from about 6 to about 7.25, most typically about 6.5 to about 7.2. including all ranges and sub ranges therebetween.

Non-limiting examples of cationic modifiers include the chloride, hydroxide, sulfate and citrate salts of calcium, magnesium, nickel, cobolt, iron, zinc and sodium. Thus, for example, calcium chloride, magnesium chloride, calcium sulfate, magnesium citrate, and the like, are exemplary cation modifiers. In a preferred embodiment, the cation modifier is selected from calcium chloride, magnesium chloride and mixtures thereof.

The cationic modifier is typically used to lower the gelling temperature during processing of the gells. The gelling temperature can be reduced to about 40° C. by adding a sufficient amount cationic modifier. When present in the compositions of the invention, the cationic modifier is present in an amount of from about 0.01% to about 2.0%, more typically from about 0.02% to about 1%, most preferably about 0.05% to about 0.5%, including all ranges and sub ranges therebetween, by weight, all weights being based on the total weight of the composition.

Pigments (g)(Optional)

The cosmetic composition of the invention may optionally include at least one pigment or dyestuff. Suitable pigments/dyes include, but are not limited to, pulverulent dyestuffs, liposoluble dyes, water-soluble dyes, and pearling agents.

The pulverulent dyestuffs may, for instance, be chosen from pigments and nacres. Useful pigments include titanium dioxide, zirconium oxide, zinc oxide, cerium oxide, iron oxide, chromium oxide, manganese violet, ultramarine blue, chromium hydrate, and ferric blue. Non-limiting examples of organic pigments include carbon black, pigments of D&C type, and lakes based on cochineal carmine, barium, strontium, calcium, and aluminum.

The nacres which may be used include, for example, mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

Representative liposoluble dyes which may be used according to the present invention include Sudan Red, DC® Red 17, DC® Green 6, beta-carotene, soybean oil, Sudan Brown, DC® Yellow 11, DC® Violet 2, DC® Orange 5, annatto, and quinoline yellow.

In an embodiment the pigment is iron oxides.

The at least one pigment/dyestuff may be present in the cosmetic composition in an amount ranging from about 5% to about 30%, more particularly from about 10% to about 25%, particularly about 20%, including all ranges and sub ranges therebetween, by weight, relative to the total weight of the compositions.

### Emollients (Optional)

The compositions of the invention may optionally include one or more emollient and/or humectants and/or moisturizers (herein “emollients”). These compounds hydrate the keratinous substrate, including the eye lids, and also provide a “wet” texture and shiny look. Emollients are known to skilled artisan. See, e.g. International Cosmetic Ingredient Dictionary and Handbook Vol. 4 (9^{th} ed. 2002), more particularly the emollients disclosed on pages 2930-2936. The disclosure of the International Cosmetic Ingredient Dictionary and Handbook Vol. 4, pages 2930-2936, is hereby incorporated by reference.

Without limitation, the emollients that may be used in the compositions of the invention include, for example: glycerin; glycerol; propylene glycol; butylene glycol; mineral oil; almond oil; castor oil; sesame oil; hydrogenated polyisobutene; butylene glycol dicaprylte dicaprate (commercially available from Sasol as Myglyol®); and mixtures thereof.

In a particular embodiment the emollient is butylene glycol.

Preferably, the compositions of the invention do not contain wax.

The emollient may be present in the composition of the invention in an amount of from about 0.1% to about 10%, preferably from about 2% to about 8%, more particularly from about 3% to about 7%, by weight, including all ranges and sub ranges therebetween, all weights being based on the total weight of the composition.

### Fillers (Optional)

The cosmetic composition disclosed herein optionally may also comprise at least one filler commonly used in the art in cosmetic compositions. The fillers may be lamellar or non-lamellar, inorganic or organic particles. Representative, non-limiting examples of these ingredients include mica, silica, kaolin, iron oxides, titanium dioxide, polyamide powders, polyamide powders, for instance Nylon® (Orgasol from Atochem), poly-alanine powders, polyethylene powders, tetrafluoroethylene polymer powders, for instance polytetrafluoroethylene (Teflon®), lauroyllysine, starch, boron nitride, hollow polymer microspheres such as those of polyvinylidene chloride/acrylonitrile, for instance Expancel® (Nobel Industries), acrylic powders such as Polytrap® (Dow Corning), polymethyl methacrylate particles and silicone resin microbeads (for example, Tospearls® from Toshiba), methylsilanol/silicate crosspolymer, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms, preferably from 12 to 18 carbon atoms, for example, zinc stearate, magnesium stearate, lithium stearate, zinc laurate, or magnesium myristate.

The fillers, if present, are present in amounts generally ranging from about 0.1% to about 25%, such as from about 1% to about 20% by weight, relative to the total weight of the composition, including all ranges and sub ranges therebetween.

### Further Additional Components (Optional)

The compositions of the present invention optionally can also include any additional ingredient or additive usually used in the field of cosmetic compositions, in particular eyeliners.

For example, these may be chosen from, for example, solvents, dispersants, antioxidants (such as pentaerythrityl tetra-di-t-butyl hydroxyhydrocinnamate), preservatives (such as for example phenoxyethanol, sodium dehydroacetate, disodium EDTA, tetrasodium EDTA, caprylyl glycol, tocopherol, potassium sorbate, alcohol denatured, and mixtures thereof), fragrances, additional thickeners or texturizers, liquid lipids/oils, additional viscosity modifiers, additional film formers, sunscreen agents, additional pigments/colorants/dyes, silica, clays, additional humectants and moisturizing agents, additional emulsifying agents (e.g. sorbitan oleate), additional structuring agents and fillers, additional surfactants, shine agents, conditioning agents, vitamins, plant extracts, additional film-formers, coalescents/plasticizers, additional pH modifiers/neutralizing agents, stabilizers, and mixtures thereof. A non-exhaustive listing of such ingredients is found in U.S. Pat. No. 7,879,316, the entire content of which is hereby incorporated by reference. Additional examples of additives may be found in the International Cosmetic Ingredient Dictionary and Handbook (9^{th} ed. 2002, and subsequent editions).

If present in the composition, these additives may constitute from 0.1% to 10%, typically from about 0.2% to about 5%, and more typically about 4%, including all ranges and sub ranges therebetween, by weight relative to the total weight of the composition.

### Cosmetic Methods

In an embodiment according to the invention, the aqueous compositions comprising a viscosity increasing system comprising at least one polysaccharide hydrocolloid and at least one viscosity increasing agent different from the polysaccharide hydrocolloid; at least one film forming polymer; and at least one liquid fatty substance; optionally at least one nonionic surfactant having an HLB greater than or equal to about 15; water; and at least one pH modifier or one cation modifier or a mixture of both; can provide a water-based gel liner having one or more the following attributes: a fresh and comfortable feel, long wear, gentle application, color intensity, and ease of removal, improved stability. Accordingly, another embodiment of the invention provides a method of making up/or enhancing the appearance of eye by applying to the eye are, topically, the composition of the present invention in a sufficient amount to make up the eye are. The compositions may be applied to the eyes, in particular the eyelids as needed, preferably once or twice daily, and then allowed to dry before subjecting to contact such as with clothing or other objects.

The compositions according to various exemplary embodiments of the invention may also have improved and/or increased ease of removability, relative to similar compositions that are not water based. In various embodiments, ease of removability relates to ease of removing the composition from the eyelids with warm (e.g. about 50° C. or higher) water. Optionally, conventional cleansing agents such as soap or make-up remover may also be used.

As such, the disclosure also relates to methods of improving at least one property of an eye liner composition selected from long wear, comfort, color intensity, water and/or oil-resistance, shine, adhesion, malleability, transfer resistance and ease of removal properties by incorporating in said eyeliner composition a polysaccharide hydrocolloid agent, such as ceratonia siliqua gum, sclerotium gum, xanthan gum, or a mixture thereof, a film forming agent and pH and/or cation modifier as described herein.

The disclosure also relates to a method of improving the stability of a liquid cosmetic composition that includes a hard gelling agent by including in said composition a polysaccharide hydrocolloid such as sclerotium gum, a film forming agent and pH and/or cation modifier as described herein.

### Method of Making

The composition according to the present disclosure may be manufactured by the known processes generally used in cosmetics. The compositions of the invention may also be made, for example, by a process comprising:
1. Providing:
   (a) at least one polysaccharide hydrocolloid and at least one viscosity increasing agent different from the polysaccharide hydrocolloid;
   (b) at least one a film forming polymer;
   (c) at least one liquid fatty substance;
   (d) optionally at least one nonionic surfactant,
   (e) providing water;
   (f) providing at least one pH adjuster or at least one cation adjuster, or a mixture of both; and
   (g) optionally a pigment; and
2. mixing until the components are uniformly dispersed.

### Container/Packaging

When the composition of the present invention is eyeliner, the composition may be packaged in an applicator product comprising a reservoir and a removable cap for closing the reservoir. The cap may, for example, form a leak-tight seal. An example of such an applicator is a pen-type applicator, such as the applicator described in U.S. Pat. Nos. 4,850,727 and 4,974,980, both of which are herein incorporated by reference.

The applicator assembly may also comprise a member for applying the composition to eyelid, wherein the applicator member allows the composition to be taken up and also allows the composition taken up to be deposited on the eyes. This applicator member can be, for example, securely fastened to the cap for leak-tight closure of the assembly.

The applicator assembly may also comprise a draining member (or drainer) for the applicator member, the draining member possibly being securely fastened to the reservoir. The applicator member may for example, be an eyeliner brush that is well known to those skilled in the art. Such a brush for instance, comprises bristles extending outwardly from and parallel to core.

Unless otherwise indicated, all numbers used in the specification and claims are to be understood as being modified in all instances by the term “about,” whether or not so stated. It should also be understood that the precise numerical values used in the specification, including the examples and claims, form additional embodiments of the invention, and are intended to include any ranges which can be narrowed to any to end points disclosed within the exemplary ranges and values provided. Efforts have been made to ensure the accuracy of the numerical values disclosed. However, any measured value can inherently contain certain errors resulting from the standard deviation found in its respective measuring technique.

### EXAMPLES

The following Examples are intended to be non-restrictive and explanatory only, with the scope of the invention being defined by the claims.

Method of Preparation of Inventive Compositions:
1. Pigments, surfactants, preservatives and water were dispersed with a homogenizing blade for 1 hr. at 60-90° C.
2. The temperature was raised to 80-90° C.
3. The polysaccharide hydrocolloid and the thickeners were added and the composition was mixed with a chopping blade until the gellant was completely hydrated. The temperature was adjusted to completely incorporate the gel.
4. Once the gel was hydrated, the temperature was cooled to 50-60° C., the dispersions of film formers were added, and the pH was adjusted with pH and cation adjusters.
5. The liquid fatty substances were then added.
6. The composition was cooled to 30-50° C. and the fillers were added.

Comparative compositions A, B, and E are commercial products as follows:

Comparator A:

Commercial anhydrous gel eyeliner containing approximately 20% more pigments than the inventive compositions. The key ingredients are: trimethylsiloxysilicate, cyclopentasiloxane, isododecane, cyclohexasiloxane, ceresin, caprylyl methicone, silica silylate, disteardimonium hectorite, trieth-oxycaprylylsilane, talc, synthetic fluorphlogopite, propylene carbonate, BHT and tin oxide. This composition has a relatively hard texture (it is sold in a pod).

Comparator B:

Commercial anhydrous gel eyeliner known for having a creamy texture. Key ingredients are: isododecane, cyclopentasiloxane, polyethylene, barium sulfate, trimethylsiloxysilicate, disteardimonium hectorite, propylene carbonate, lecithin, phenyl trimethicone, ethylhexylglycerin, hydrogenated polyisobutene and methicone. This composition has a relatively soft texture and is also available in a pod.

Comparator E:

Commercial water based liquid eyeliner containing pigments at level greater than 15%, styrene/acrylates/ammonium methacrylate copolymer, polyester-5, pigment, propylene glycol, xanthan gum and polysorbate 80.

TABLE 1
Anionic Polysaccharide and Galactomannan

| | Comparative D | Ex. 1 |
|---|---|---|
| INCI US | (Wt % actives) | (Wt. % actives) |
| styrene/acrylates/ammonium | 7.37625 | 7.37625 |
| methacrylate copolymer (b) | | |
| iron oxides (g) | 20 | 20 |
| divinyldimethicone/dimethicone | 1.8 | 1.8 |
| copolymer (b) | | |
| methylsilanol/silicate crosspolymer | | 1.5 |
| (filler) | | |
| phenyl trimethicone (c) | 3 | 3 |
| citric acid (f) | | 0.15 |
| sodium laureth sulfate | 0.16875 | 0.16875 |
| (anionic emulsifier) | | |
| sodium citrate (f) | 0.004 | 0.004 |
| Tocopherol (preservative) | 0.0004 | 0.0004 |
| dimethicone/peg-10/15 crosspolymer (c) | 0.5 | 0.5 |
| ppg-7 | 0.09375 | 0.09375 |
| (emulsifier) | | |
| peg-10 dimethicone (c) | 0.5 | 0.5 |
| butylene glycol | 4.21875 | 4.21875 |
| (plasticizer) | | |
| dipropylene glycol | 0.01 | 0.01 |
| (plasticizer) | | |
| xanthan gum (a)(ii) | 0.5 | 0.5 |
| caprylyl glycol | 0.4275 | 0.4275 |
| (preservative) | | |
| phenoxyethanol | 0.664625 | 0.664625 |
| (preservative) | | |
| ammonium hydroxide (f) | 0.00625 | 0.00625 |
| sorbitan oleate | 0.0125 | 0.0125 |
| (emulsifier) | | |
| disodium EDTA | 0.198 | |
| (preservative) | | |
| potassium sorbate | 0.01125 | 0.01125 |
| (preservative) | | |
| styrene/acrylates copolymer (b) | 2.84375 | 2.84375 |
| acrylamide/sodium | 0.2 | 0.2 |
| acryloyldimethyltaurate | | |
| copolymer (a)(iii) | | |
| Isohexadecane | 0.105 | 0.105 |
| (solvent) | | |
| sodium dehydroacetate | 0.2 | 0.2 |
| (preservative) | | |
| Dimethicone (c) | 1.4856 | 1.4856 |
| Tetrasodium EDTA (preservative) | 0.028125 | 0.028125 |
| alcohol denat. (solvent) | 3 | 3 |
| c12-13 pareth-3 | 0.06 | 0.06 |
| (emulsifier) | | |
| c12-13 pareth-23 | 0.084 | 0.084 |
| (emulsifier) | | |
| Silica | | 0.5 |
| (filler) | | |
| Water (e) | 51.2665 | 47.3145 |
| ceratonia siliqua gum (a)(i) | 0.5 | 0.5 |
| PTFE | | 2 |
| (filler) | | |
| polysorbate 80 (d) | 0.535 | 0.535 |
| sodium hyaluronate (a)(ii) | 0.2 | 0.2 |

TABLE 2
Homopolysaccharide

| | Comparator C | Ex. 2 |
|---|---|---|
| INCI US | (Wt % Actives) | (Wt % Actives) |
| styrene/acrylates/ammonium | 7.37625 | 7.37625 |
| methacrylate copolymer (b) | | |
| iron oxides (g) | 20 | 20 |
| divinyldimethicone/dimethicone | 1.8 | 1.8 |
| copolymer (b) | | |
| phenyl trimethicone (c) | 3 | 3 |
| citric acid (f) | | 0.2 |
| sodium laureth sulfate | 0.16875 | 0.16875 |
| (emulsifier) | | |
| sodium citrate (f) | 0.004 | 0.004 |
| tocopherol | 0.0004 | 0.0004 |
| (preservative) | | |
| dimethicone/peg-10/15 crosspolymer (c) | 0.5 | 0.5 |
| PPG-7 | 0.09375 | 0.09375 |
| (emulsifier) | | |
| butylene glycol | 4.21875 | 4.21875 |
| (plasticizer) | | |
| dipropylene glycol | 0.01 | 0.01 |
| (plasticizer) | | |
| xanthan gum (a)(ii) | | 1 |
| (a)(ii) | | |
| caprylyl glycol | 0.4275 | 0.4275 |
| (preservative) | | |
| phenoxyethanol | 0.664625 | 0.664625 |
| (preservative) | | |
| ammonium hydroxide (f) | 0.00625 | 0.00625 |
| sorbitan oleate | 0.025 | 0.025 |
| (emulsifier) | | |
| disodium EDTA | 0.198 | 0.198 |
| (preservative) | | |
| potassium sorbate | 0.01125 | 0.01125 |
| (preservative) | | |
| styrene/acrylates copolymer (b) | 2.84375 | 2.84375 |
| acrylamide/sodium | 0.4 | 0.4 |
| acryloyldimethyltaurate | | |
| copolymer (a)(iii) | | |
| sclerotium gum (a)(i) | 2 | 2 |
| isohexadecane | 0.21 | 0.21 |
| (solvent) | | |
| sodium dehydroacetate | 0.2 | 0.2 |
| (preservative) | | |
| dimethicone (c) | 1.4856 | 1.4856 |
| tetrasodium EDTA | 0.028125 | 0.028125 |
| (preservative) | | |
| alcohol denat. | 3 | 3 |
| (preservative) | | |
| c12-13 pareth-3 | 0.06 | 0.06 |
| (emulsifier) | | |
| c12-13 pareth-23 | 0.084 | 0.084 |
| (emulsifier) | | |
| Water (e) | 47.914 | 46.714 |
| polysorbate 80 (d) | 3.07 | 3.07 |
| sodium hyaluronate (a)(ii) | 0.2 | 0.2 |

TABLE 3
Anionic Polysaccharide/Neutral Polysaccharide/Synthetic
Thickener

| | Ex. 3 | Ex. 4 |
|---|---|---|
| INCI US | (Wt % Actives) | (Wt % Actives) |
| styrene/acrylates/ammonium | | 7.37625 |
| methacrylate copolymer (b) | | |
| Polymethylsilsesquioxane (b) | 9 | |
| iron oxides (g) | 30 | 20 |
| divinyldimethicone/dimethicone | | 1.8 |
| copolymer (b) | | |
| phenyl trimethicone (c) | 3 | 3 |
| agarose (a)(i) | | 1 |
| sodium laureth sulfate | | 0.16875 |
| (emulsifier) | | |
| sodium citrate (f) | | 0.004 |
| Tocopherol | | 0.0004 |
| (preservative) | | |
| ammonium acryloyldimethyltaurate/ | 0.184 | |
| steareth-8 methacrylate copolymer | | |
| (a)(iii) | | |
| dimethicone/peg-10/15 crosspolymer (c) | | 0.5 |
| PPG-7 | | 0.09375 |
| (emulsifier) | | |
| calcium chloride (f) | 0.1 | |
| peg-10 dimethicone (c) | 1.5 | |
| butylene glycol | 3.9 | 4.21875 |
| (plasticizer) | | |
| dipropylene glycol | | 0.01 |
| (plasticizer) | | |
| xanthan gum | 0.3 | |
| caprylyl glycol | 0.3 | 0.4275 |
| (preservative) | | |
| Phenoxyethanol | 0.5 | 0.664625 |
| (preservative) | | |
| ammonium hydroxide (f) | | 0.00625 |
| sorbitan oleate | | 0.025 |
| (emulsifier) | | |
| disodium EDTA | | 0.198 |
| (preservative) | | |
| potassium sorbate | | 0.01125 |
| styrene/acrylates copolymer (b) | | 2.84375 |
| acrylamide/sodium | | 0.4 |
| acryloyldimethyltaurate | | |
| copolymer (a)(iii) | | |
| Isohexadecane | | 0.21 |
| (solvent) | | |
| sodium dehydroacetate | 0.2 | 0.2 |
| (preservative) | | |
| glycerin | 4 | |
| (emollient) | | |
| dimethicone (c) | 2.2 | 1.4856 |
| dimethiconol (c) | 0.3 | |
| tetrasodium EDTA | 0.198 | 0.028125 |
| (preservative) | | |
| alcohol denat. | | 3 |
| (preservative) | | |
| c12-13 pareth-3 | | 0.06 |
| (emulsifier) | | |
| c12-13 pareth-23 | | 0.084 |
| (emulsifier) | | |
| gellan gum (a)(i) | 0.8 | |
| t-butyl alcohol | 0.006 | |
| (solvent) | | |
| water (e) | 32.312 | 48.914 |
| polysorbate 80 | | 3.07 |
| (d) | | |
| sodium hyaluronate (a)(ii) | 0.2 | 0.2 |
| polyethylene | 2 | |
| (filler) | | |
| vinyl dimethicone/methicone | 2.5 | |
| silisesquioxane crosspolymer | | |
| (filler) | | |
| Silica | 0.5 | |
| (filler) | | |
| caprylyl methicone (c) | 2.5 | |
| bis-diglyceryl polacyladipate-2 (c) | 2 | |

The viscosity and pH of inventive and comparative compositions were measured according to Methods A and B above. The measurements were made on aged formulas as noted. The results of these measurements are reported in Table 4 below.

TABLE 4

| | | | Apparent | | |
|---|---|---|---|---|---|
| | | | Viscosity (Pa · s) | | |
| | | | Method A | | |
| Example/Comparator | Aged | | (spindle) | | pH Method B |
| Comparator C | 16 | months | 8.7 | (4) | 7.65 |
| Comparator D | 15 | months | 4.7 | (4) | 7.45 |
| Ex. 1 | 12 | months | 16.8 | (4) | 7.10 |
| Ex. 2 | 5 | months | 10.9 | (4) | 6.96 |
| Ex. 3 | 4 | months | 32.5 | (5) | 6.82 |
| Ex. 4 | 16 | months | 1.49 | (3) | 7.53 |
| Ex. 5 | 15 | months | 12.8 | (4) | 7.28 |

Table 4 illustrates the trend in viscosity and pH of the water-based inventive compositions compared to comparative compositions C and D, all of which compositions are gels. As is shown in Table 4, the compositions of the invention (Examples 1 and 2), even while being water-based, retain their viscosity over time and have higher (more desirable viscosities) than comparable comparative compositions C (which corresponds to Ex 2 but has a pH higher than 7.2) and D (corresponding to Ex 1 again with a pH higher than 7.2. Table 4 also shows that depending on the viscosity system chosen, different product features are available.

Texture and Spreadability Analysis

The texture and spreadability of the inventive compositions and of Comparators A-D were assessed using Methods C and D, described above. The results of this assessment are provided below in Table 5.

TABLE 5

| | | Texture Analysis (Method C) | | | Spreadability | |
|---|---|---|---|---|---|---|
| | | (n = 4) | | | (n = 2-3, Method D) | |
| | | | Average | Average | Mean Max | Mean area |
| | | Average | Work of | Resistance | Force | “Work of |
| | | Firmness ± | Penetration ± | to Probe ± | “Firmness’ ± | Shear” ± |
| | | stdevp | stdevp | stdevp | stdevp | stdevp |
| Example | Aged | (grams, g) | (g/seccond) | (g/second) | (grams, g) | (g/seccond) |
| Comparator A | unknown | 227.06 ± | 783.41 ± | −284.78 ± | n.d. | n.d. |
| | | 1.72 | 8.35 | 9.38 | | |
| | | (n = 2) | (n = 2) | (n = 2) | | |
| Comparator B | unknown | n.d. | n.d. | n.d. | 757.97 ± | 736.14 ± |
| | | | | | 14.44 | 9.08 |
| | | | | | (n = 2) | (n = 2) |
| Comparator C | 16 months | 26.81 ± | 85.41 ± | −27.58 ± | 1186.91 ± | 1408.27 ± |
| | | 0.59 | 2.24 | 0.68 | 68.71 | 82.30 |
| | | | | | (n = 3) | (n = 3) |
| Comparator D | 15 months | Too low | Too low | Too low | 386.96 ± | 340.05 ± |
| | | to be | to be | to be | 8.90 | 5.27 |
| | | determined | determined | determined | | |
| Ex. 1 | 12 months | 21.09 ± | 62.73 ± | −30.77 ± | 1529.75 ± | 1481.02 ± |
| | | 0.83 | 1.88 | 0.73 | 70.77 | 30.34 |
| | | | | | (n = 3) | (n = 3) |
| Ex. 2 | 5 months | 16.23 ± | 50.92 ± | −24.22 ± | 1020.46 ± | 1061.43 ± |
| | | 0.23 | 0.83 | 0.22 | 27.04 | 44.20 |
| Ex. 3 | 4 months | 20.44 ± | 61.46 ± | −53.03 ± | 1840.33 ± | 1602.08 ± |
| | | 0.87 | 0.99 | 1.65 | 30.62 | 48.83 |
| | | | | | (n = 3) | (n = 3) |
| Ex. 4 | 16 months | n.d. | n.d. | n.d. | n.d. | n.d. |
| Ex. 5 | 15 months | 10.84 ± | 33.29 ± | −36.11 ± | 891.46 ± | 735.29 ± |
| | | 0.08 | 0.12 | 0.67 | 1.32 | 19.74 |
| | | | | | (n = 3) | (n = 3) |

n.d. = not determined;
n = 4-8.
Note
data compiled from experiments run according to Method D and E at different times.
Results are reported as an average ± standard deviation p.

As determined using Method D, the water-based gel eyeliners of inventive compositions of Ex 2-4 have a higher average firmness and spreadability than commercial anhydrous Comparator B (sold in a jar). In addition, as determined using Method C, all of the inventive compositions have softer textures as determined by texture analysis using a 6 mm probe, in comparison to a harder commercial anhydrous gel eyeliner (Comparator A).

Acidification of Comparator C with 0.2% citric acid (delta pH −0.6) and addition of 1% Xanthan gum to Comparator C (which is Example 3) resulted in a visibly thicker formula with a slightly higher high shear viscosity as determined by Method A (Table 4) but did not result in a harder texture (Table 5). Instead, both Comparator C and Ex. 3 showed comparable peak force firmness in the Spreadability test, but addition of xanthan gum in Ex. 3 did, however, unexpectedly lower the “Work of Shear.”

Acidification of Example 1 with 0.15% Citric Acid increased both the “Firmness” and the “Work of Shear” of Example 1 versus Comparator D (Method D). Texture analysis of Comparator D could not be performed as formula was to fluid and did not have a measurable peak force hardness (Method C).

The composition of Ex. 3 (containing gellan gum) showed the highest “Firmness” and “Work of Shear” values than all examples in the Spreadability test (Method D). However, contrary to expectation, the composition of Ex. 3, did not have the give the hardest texture according to texture analysis using Method C.

The stability of a representative formula of the invention (Ex. 5) was compared to the composition of Comparator E using the centrifugal stability test of Method E (described above). The results of this assessment are reported below in Table 6.

TABLE 6

| | | | Total | | Film | | |
|---|---|---|---|---|---|---|---|
| | | Centri- | height (hₜ) of | Pigment/ | former | upper | |
| | | fugation | filled tube | pellet | phase | phase | % |
| Example | Trial | stability | (mm) | (hₚ) (mm) | (hₒ, mm) | (hₛ, mm) | Demixing |
| Comparator E | 1 | no | 77 | 34 | 16 | 27 | 56% |
| Ex. 5 | 1 | yes | 77 | 0 | 0 | 0 | 0% |

As is shown in Table 6, the composition of Ex. 5 did not separate out and remained stable even during centrifugation (0% demixing). The composition of Ex. 5 was liquid as is confirmed by the viscosity in Table 4. When subjected to the same centrifugation test, however, the composition of Comparator E separated into three distinct and very visible phases: a precipitated pigment pellet and two separate liquid phases. This shows that the aqueous compositions of the invention are stable and do not separate out over time, even when a hard polysaccharide hydrocolloid (agarose) is used.

What is claimed is:

1. In an embodiment, the invention relates to a water-based gel cosmetic composition comprising:
(a) a viscosity increasing system comprising (i) at least one polysaccharide hydrocolloid, and (ii) at least one viscosity increasing agent different from (a)(i);
(b) at least one film forming polymer;
(c) at least one liquid fatty substance;
(d) optionally at least one nonionic surfactant having an HLB greater than or equal to about 15;
(e) water;
(f) at least one pH adjuster or at least one cation adjuster, or a mixture of both; and
(g) optionally a pigment.

2. The composition of claim 1 having a pH less than about 7.5.

3. The composition of claim 2 having a pH less than about 7.2.

4. The composition of claim 3 wherein the ratio of the least one polysaccharide hydrocolloid (a)(i) to the at least on viscosity increasing agent (a)(ii) is less than or equal to about 4:1.

5. The composition of claim 4 having a viscosity greater than or equal to about 10 Pa s.

6. The composition of claim 5 comprising a nonionic surfactant having an HLB greater than or equal to about 15.

7. The composition of claim 6 wherein the at least one polysaccharide hydrocolloid (a)(i) is present in an amount from about 0.5% to about 3.5% by weight relative to the weight of the composition.

8. The composition of claim 7 wherein the at least one viscosity increasing agent (a)(ii) is present in the composition in an amount from about 0.1% to about 3% by weight relative to the weight of the composition.

9. The composition of claim 8 wherein the viscosity increasing system is present in an amount of from about 0.5% to about 3.5% by weight relative to the weight of the composition.

10. The composition of claim 9 wherein the film forming polymer (b) is present in an amount from about 0.5% to about 20%

11. The composition of claim 10 wherein the liquid fatty substance (c) is present in an amount from about 1% to about 20% by weight relative to the weight of the composition.

12. The composition of claim 11 wherein the nonionic surfactant (d) is present in an amount from about from about 0.1% to about 5% by weight relative to the weight of the composition.

13. The composition of claim 12 wherein the pH adjuster (f) is present in an amount from about 0.01% to about 2.0% by weight relative to the weight of the composition.

14. The composition of claim 13 wherein the polysaccharide hydrocolloid (a)(i) is selected from a homopolysaccharide, pseudoplastic anionic polysaccharide, neutral polysaccharide, and a mixture thereof.

15. The composition of claim 14 wherein the viscosity increasing agent (a)(ii) is selected from an anionic polysaccharide, a synthetic thickener, and mixtures thereof.

16. The composition of claim 15 wherein the film forming polymer is selected from at least one styrene/acrylates copolymer, a silicone copolymer, and mixtures thereof.

17. The composition of claim 15 wherein the viscosity increasing agent (a)(ii) is selected from an anionic polysaccharide.

18. The composition of claim 17 wherein the polysaccharide hydrocolloid (a)(i) is selected from sclerotium gum, carrageenan, gellan gum, galactomannan gum, glucomannan gum, agarose, and mixtures thereof.

19. Water-based gel cosmetic composition comprising:
(a) from 0.5% to about 3.5%a viscosity increasing system comprising: (i) from about 0.6% to about 3% of at least one polysaccharide hydrocolloid selected from agarose, and (ii) from about 0.2% to about 2.5% of at least one viscosity increasing agent selected from sodium hyaluronate;
(b) from about 4% to about 18% of at least one film forming polymer selected from styrene/acrylates copolymer, styrene/acrylates/ammonium methacrylate copolymer, and mixtures thereof;
(c) at least one liquid fatty substance;
(d) optionally at least one nonionic surfactant having an HLB greater than or equal to about 15;
(e) water;
(f) at least one pH adjuster or at least one cation adjuster, or a mixture of both; and
(g) optionally a pigment;
said composition having a pH less than about 7.55 and a viscosity less than about 2 Pa·s.

20. A kit for making up the eyes comprising a pen-type applicator containing the composition of claim 1.

21. A method of making up or enhancing the appearance of the eye by applying to the eyes, topically, a composition comprising:
(a) a viscosity increasing system comprising (i) at least one polysaccharide hydrocolloid, and (ii) at least one viscosity increasing agent different from (a)(i);
(b) at least one film forming polymer;
(c) at least one liquid fatty substance;
(d) optionally at least one nonionic surfactant having an HLB greater than or equal to about 15;
(e) water;
(f) at least one pH adjuster or at least one cation adjuster, or a mixture of both; and
(g) optionally a pigment;

22. A method of improving at least one property of an eye liner composition selected from long wear, comfort, color intensity, water and/or oil-resistance, shine, adhesion, malleability, transfer resistance and ease of removal properties by incorporating in said eyeliner composition a polysaccharide hydrocolloid, a film forming agent and pH and/or cation modifier.

23. A method of making a composition according to claim 1 comprising:
I. Providing
(a) at least one polysaccharide hydrocolloid and at least one viscosity increasing agent different from the polysaccharide hydrocolloid;
(b) at least one a film forming polymer;
(c) at least one liquid fatty substance;
(d) optionally at least one nonionic surfactant,
(e) providing water;
(f) providing at least one pH adjuster or at least one cation adjuster, or a mixture of both; and
(g) optionally a pigment; and
II. mixing until the components are uniform.
